(19) United States
(12) Patent Application Publication
(10) Pub. No.: US 2022/0072213 A1
(43) Pub. Date: Mar. 10, 2022

(54) Systems and Methods to Facilitate the Non-Surgical Management of Obstructive Urolithiasis

(71) Applicant: Thormed Innovation, LLC, Dallas, TX (US)

(72) Inventor: Gregory Robert Thoreson, Dallas, TX (US)

(73) Assignee: Thormed Innovation, LLC

(21) Appl. No.: 17/399,311

(22) Filed: Aug. 11, 2021

Related U.S. Application Data
(60) Provisional application No. 63/076,409, filed on Sep. 10, 2020.

(51) Int. Cl. A61M 1/00

(52) U.S. Cl. CPC A61M 1/77; A61M 1/743; A61M 1/70; A61M 2210/1085; A61M 1/69; A61M 2210/1082; A61M 2210/1089; A61M 1/86

(57) ABSTRACT

The present invention discloses systems and methods for use in the treatment and management of obstructive urolithiasis. A coupling device between a nephrostomy tube and external fluid and control connections may be used in some embodiments of the present invention. This coupling device may provide a quick connection between the nephrostomy tube and a reservoir for irrigation and a collection container for drainage of the kidney. In some embodiments, a triple lumen nephrostomy tube may be used to house an irrigation catheter for irrigation of the patient's kidneys and a ureteral modulation wire and enable drainage of fluid from the kidneys. The ureteral modulation wire may be inserted into the patient's ureter through the kidney in conjunction with the nephrostomy tube. A harness system is also disclosed that may be used in conjunction with the coupling device and the triple lumen nephrostomy tube to enable a patient to independently control irrigation and drainage of the kidney.

### PRIORITY CLAIM

This application claims priority to and is filed as the parent non-provisional application for provisional U.S. patent application Ser. No. 63/076,409 that was filed on Sep. 10, 2020.

### TECHNICAL FIELD

The present invention relates generally to systems and devices that assist with the non-surgical treatment of obstructive urolithiasis or kidney stones, and more specifically, to devices that will enable a patient to independently self-administer therapy for the treatment of kidney stones, including devices that alleviate medical conditions that are the result of kidney stones and enable the patient to pass kidney stones.

### BACKGROUND OF THE INVENTION

Urolithiasis or the formation of calcifications in the kidney or ureter (kidney stones) is a highly prevalent condition with an incidence of 1-15%. Total estimated expenditures for managing urolithiasis was $2.1 billion in 2000 and $10.3 billion in 2006, indicating a significant increase. With conditions like diabetes and obesity on the rise and population growth, annual expenditures for managing urolithiasis is expected to continue to grow. Unless complicating factors are present at the time of diagnosis, contemporary management of urolithiasis encourages immediate implementation of conservative measures, which can last for up to six weeks. If complicating factors are present, surgical decompression with the placement of a ureteral stent or percutaneous nephrostomy tube is recommended.

Ureteral stents are self-retaining drainage tubes placed within the ureter that allow for equilibrium of hydrostatic pressure between two systems, specifically the upper urinary system (i.e., kidneys) and the lower urinary system (i.e., bladder). Under normal circumstances, these two systems do not transfer pressure because of the unidirectional antireflux mechanism associated with the ureterovesicular junction that prevents the transference of pressure from the bladder into the kidney during micturition. However, in the presence of acute renal colic (obstruction of the urinary tract), the relative pressure of the bladder is lower than that of the kidney, which is why stent placement is effective in lowering the pressure within the kidney thereby facilitating drainage of an obstructed system. Conversely, when pressures within the bladder are increased during a micturition sequence, that pressure is transferred to the kidney, bypassing the endogenous anti-reflux system of the ureterovesicular junction, resulting in renal colic during urination with a ureteral stent in place. Other common symptoms of patients with indwelling ureteral stents are urinary frequency and bladder spasms resulting from the constant mechanical irritation of the trigone of the bladder.

Percutaneous nephrostomy tube placement has distinct advantages over ureteral stent placement for managing an obstructed urinary system. Costs are reduced by a factor of two, there is a higher rate of spontaneous stone passage when adjusted for stone size and location, and there is a significant improvement in symptoms, including dysuria, urinary urgency, frequency, and discomfort, quality of life, anxiety, depression, and the amount of pain medication required. However, there are distinct disadvantages as well. “Tube symptoms” appear after initial placement, including personal hygiene issues and impaired mobility, but these commonly resolve over time as the patient becomes more accustomed to the new hardware.

Conservative management of obstructive urolithiasis generally consists of pain control, encouraged electrolyte balanced fluid repletion, and medical expulsive therapy for up to six weeks. The intent of medical expulsive therapy is to improve outcomes during the conservative management of obstructive urolithiasis. Randomized trials implementing medical expulsive therapy have demonstrated conflicting results without conclusive benefit. However, other studies have demonstrated that, with appropriately selected patients, there may be a benefit in patients with obstructive urolithiasis greater than 5 mm.

A shortage of urologists is expected in the near future, and devices that serve as a surrogate for the care that a urologist would provide in real time would prove extremely useful and valuable. Improvement in comfort of patients that undergo percutaneous drainage of their kidneys is also desired. Cancer patients who are not candidates for the reconstruction of their urinary system and are dependent on long-term percutaneous drainage of the kidney would also benefit from the development of improved devices.

Due to the complications and drawbacks described above with current treatments for obstructive urolithiasis, different devices, apparatuses, systems, and methods are desired for improved outcomes. Further, novel devices or systems that can demonstrate improved outcomes with reduced costs will lead to adaptation by healthcare providers and institutions.

### BRIEF SUMMARY OF THE INVENTION

The present invention includes novel medical devices for use in with the treatment and management of obstructive urolithiasis. These novel devices may (1) decompress the kidney only when the intrarenal pressures are elevated, (2) irrigate each calyx of the kidney at low pressures over a long period of time in an ambulatory setting, (3) enable patients to independently self-administer the therapy safely and comfortably, and (4) enable the ureter to gently expand over time to accommodate larger stone fragments while eliminating arrhythmic contractions of the ureter.

In one embodiment of the present invention, a coupling device between a nephrostomy tube and external fluid and control connections is disclosed. This coupling device may provide a quick connection between a nephrostomy tube and a reservoir for irrigation and a collection container for drainage of the kidney. With this coupling device, a patient may better control attachment and detachment to the nephrostomy tube, thereby allowing irrigation and drainage of the kidneys. An irrigation valve within the coupling device may control the timing and amount of irrigation, and a drainage valve may interface to the container for drainage of the kidney. An irrigation catheter may be used to irrigate the kidneys. The coupling device may also contain an outflow pressure regulator to monitor the drainage pressure from the nephrostomy tube to control the irrigation and drainage of the kidneys in response to the pressure in the kidney. The coupling device may adhere to the skin of the patient to enable quick and simple connection between the nephrostomy tube and the external fluid and control connections.

In another embodiment of the present invention, a ureteral modulation wire is disclosed. This ureteral modulation wire may be inserted into the patient's ureter through the kidney in conjunction with the nephrostomy tube. The ureteral modulation wire may include a first set of electrodes for detecting ureteral peristalsis and a second set of electrodes for transmission of electrical current to stimulate the ureter. The transmission of electrical current may be used to induce ureteral peristalsis or paralyze the ureter preventing ureteral peristalsis. In addition to these electrodes, the ureteral modulation wire may include reinforced tubing that is capable of radial expansion. Prolonged expansion of this tubing may increase the diameter and pliability of the ureter, thus allowing the passage of solid objects through the ureter. The coupling device may also include connection to the ureteral modulation wire for external control.

In some embodiments, a triple lumen nephrostomy tube is disclosed to house the irrigation catheter, the ureteral modulation wire, and enable drainage of fluid from the kidneys. A first channel within the outer tube of the triple lumen nephrostomy tube may be used to irrigate the kidney. An irrigation catheter may fit through the first channel and include an irrigation catheter tip for opening and closing the irrigation catheter. A second channel within the outer tube of the triple lumen nephrostomy tube may be used to house the ureteral modulation wire. The ureteral modulation wire may fit through this second channel and include a wire tip to ensure proper placement in the patient. The irrigation catheter tip and the ureteral modulation wire tip may be variable buoyancy tips according to certain embodiments. This may allow the irrigation catheter to more easily move through the kidneys of the patient and allow the ureteral modulation wire to float within the bladder of the patient.

In another embodiment of the present invention, a harness system may be used in conjunction with the coupling device and the triple lumen nephrostomy tube to enable a patient to independently control irrigation and drainage without significant assistance from healthcare personnel. The harness system may include straps and buckles to hold the necessary components. An irrigation unit may provide the water supply for the irrigation catheter and a drainage unit may collect the urine drainage from the kidney. After the triple lumen nephrostomy tube is inserted in the patient, the coupling device may be used to interface the components of the harness system to the triple lumen nephrostomy tube. More specifically, a connection from the irrigation unit to the irrigation catheter may allow water to irrigate the patient's kidney, wherein an irrigation valve within the coupling device may control said irrigation. A drainage valve may control the fluid leaving the kidney and entering into the drainage unit. An outflow pressure regulator within the coupling device may be used to monitor the drainage pressure from the nephrostomy tube to control the irrigation and drainage of the kidneys in response to the pressure in the kidney. Through the coupling device, the harness system may further enable external control of the ureteral modulation wire. Quick connections between the irrigation unit and the coupling device, the drainage unit and the coupling device, and the triple lumen nephrostomy tube and the coupling device may allow the patient to easily connect and disconnect the harness system. The harness system may improve the independence of a patient for the non-surgical treatment of obstructive urolithiasis.

### BRIEF DESCRIPTION OF THE DRAWINGS

For a more complete understanding of the present invention, reference is now made to the following descriptions taken in conjunction with the accompanying drawing, in which:

FIG. 1 shows an internal anterior view of an upper and a lower urinary system of a patient;

FIG. 2 shows an internal anterior view of an upper and a lower urinary system of the patient and corresponding placement of novel devices for the treatment of obstructive urolithiasis according to certain embodiments of the present invention;

FIG. 3 shows an internal anterior view of anterior view of an upper urinary system of the patient and corresponding placement of novel devices for the treatment of obstructive urolithiasis according to certain embodiments of the present invention;

FIG. 4 shows a cross-section view of a triple lumen nephrostomy tube according to certain embodiments of the present invention;

FIG. 5 shows a cross-section view of a left-sided coupling device between a triple lumen nephrostomy tube and external fluid and control connections according to certain embodiments of the present invention;

FIG. 6 shows multiple views of a connection interface for a coupling device between a triple lumen nephrostomy tube and external fluid and control connections according to certain embodiments of the present invention;

FIG. 7 shows multiple views of an alternative connection interface for a coupling device between a triple lumen nephrostomy tube and external fluid and control connections according to certain embodiments of the present invention;

FIG. 8 shows a harness system for enabling a patient to efficiently transport and access external fluid and control connections to a triple lumen nephrostomy tube according to certain embodiments of the present invention;

FIG. 9 shows a ureteral modulation unit according to certain embodiments of the present invention;

FIG. 10 shows a ureteral modulation wire according to certain embodiments of the present invention; and

FIG. 11 shows an irrigation catheter according to certain embodiments of the present invention.

### DETAILED DESCRIPTION OF THE INVENTION

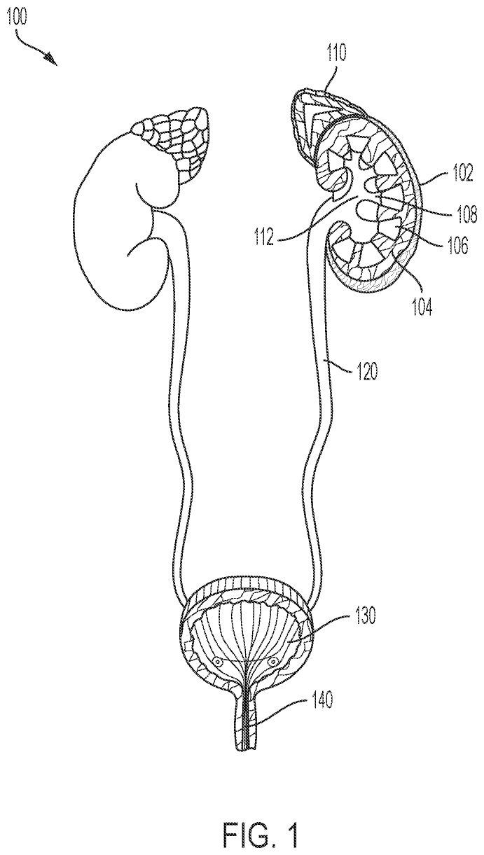

FIG. 1 illustrates an internal anterior view of an upper and a lower urinary system of a patient 100. The reference numerals are shown on the left side of the patient, but the right side of the patient has the same upper and lower urinary tract or system. A left kidney 102 connects to a bladder 130 through a ureter 120. As urine leaves the kidney 102, the ureter 120 transports the urine to the bladder 130. From there, a urethra 140 then transports the urine out of the body of the patient. The kidney 102 and the ureter 120 are considered the upper urinary tract or system, and the bladder 130 and the urethra 140 are considered the lower urinary tract or system. Adrenal glands 110 are located at the top of the kidneys 102. Adrenal glands 110 produce hormones that regulate metabolism, immune system, blood pressure, response to stress, and other essential functions.

The kidney 102 filters the patient's blood, removes the waste, and excretes the waste in the form of urine. The kidney 102 is made up of a cortex 104 which is outer portion of the kidney 102 and a renal medulla 106, which is a darker inner portion that consists of pyramid structures throughout the kidney. The cortex 104 and the renal medulla 106 make up the functional tissue of the kidney 102. A calyx 108 (minor and major) collects the urine excreted from the pyramid structures of the renal medulla 106. The portions of the calyx 108 converge at a renal pelvis 112, which connects the renal medulla 106 to the ureter 120.

The ureter 120 then passes the urine to the bladder 130, where the urine is stored until excreted by the patient. The bladder 130 is expandable, so when urine accumulates in the bladder 130 it will fill up like a balloon. Nerves in the bladder 130 wall detect this expansion and signal to the brain that the patient's bladder 130 is full. Then the patient expels the urine through the urethra 140. This is how the blood is filtered and the wastes are excreted by the urinary tract or system.

If more crystal-forming substances are formed in the kidney 102 than the kidney 102 can dilute with urine, then crystals or kidney stones may form. Primarily, kidney stones are developed from calcium, oxalate, and/or uric acid. A patient's kidney 102 may also lack the ability to produce the necessary substances to prevent crystals from sticking together, thereby allowing these smaller crystals to create larger crystals. There are four types of kidney stones, (1) calcium stones (calcium oxalate), which are the most common, (2) struvite stones, which develop in response to a urinary tract infection, (3) uric acid stones, and (4) cystine stone, which develop from a hereditary disease. After passing from the kidneys to the ureter, these kidney stones can lead to numerous adverse medical conditions, including obstructions in the ureter or inflammation in the ureter. Thus, medical solutions that can prevent or reduce the calcification of kidney stones in the kidneys and/or enable the ureter to more easily pass a kidney stone can provide significant relief to a patient.

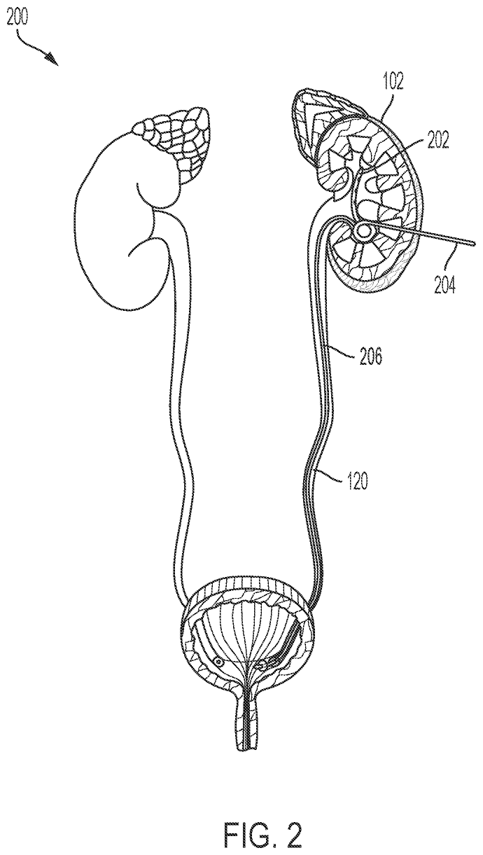

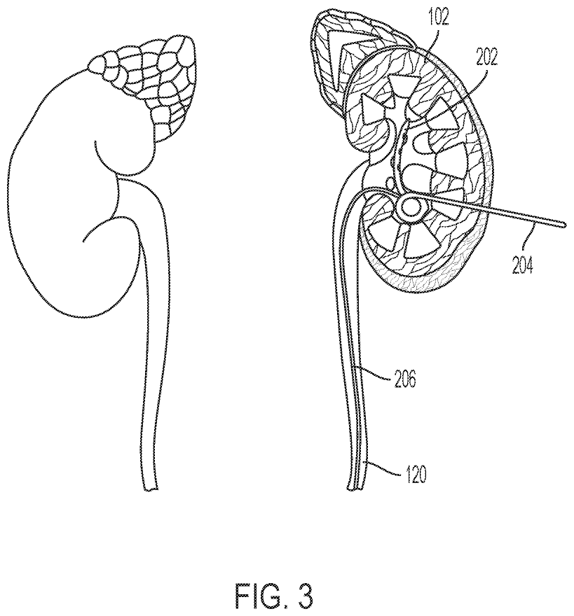

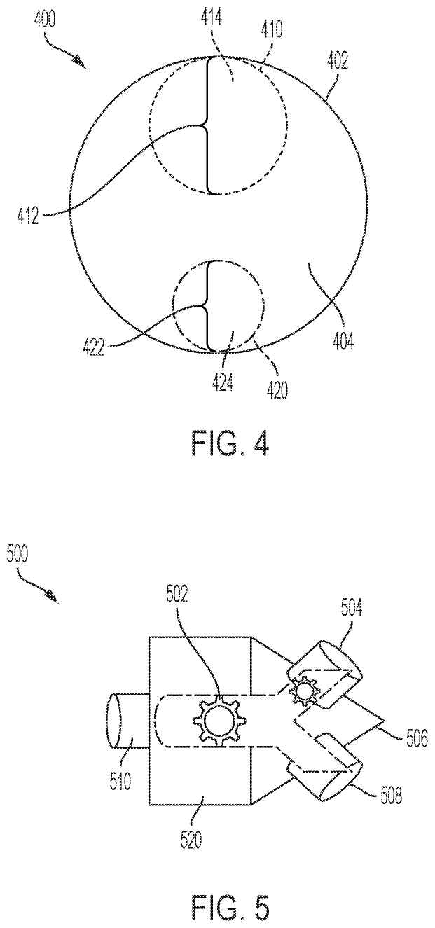

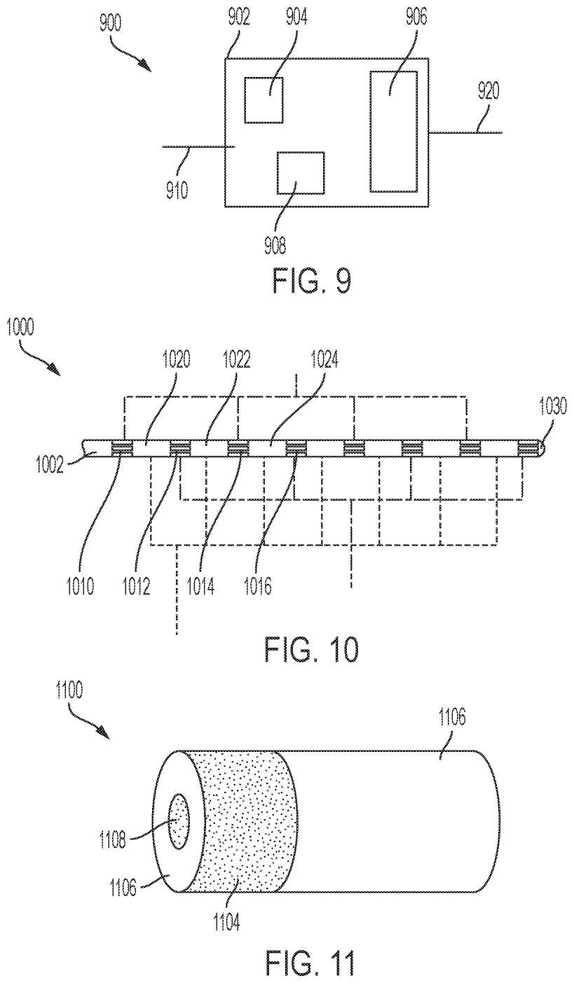

FIG. 2 shows an internal anterior view of an upper and a lower urinary system of the patient and corresponding placement of novel devices for the treatment of obstructive urolithiasis according to certain embodiments of the present invention. FIG. 3 shows an internal anterior view of an upper urinary system of the patient and corresponding placement of novel devices for the treatment of obstructive urolithiasis according to certain embodiments of the present invention. Various components of the novel solution of the present invention will be further described herein, but FIGS. 2-3 illustrate the placement of these components in the kidney 102 and ureter 120. A triple lumen nephrostomy tube 204 is deployed in the patient. An irrigation catheter 202 enters the kidney 102 within the triple lumen nephrostomy tube 204 and is designed to irrigate the renal pelvis and calyx of the kidney 102. The irrigation catheter 202 may be designed to be flexible to enable it to move throughout this portion of the kidney 102. FIGS. 4 and 11 further illustrate features of the irrigation catheter 202. A ureteral modulation wire 206 enters the kidney 102 and then the ureter 120 within the triple lumen nephrostomy tube 204 as well. The ureteral modulation wire 206 may be designed to be flexible to enable it to move down the ureter 120 towards the bladder. FIG. 2 shows the ureteral modulation wire 206 entering the bladder. FIGS. 9 and 10 further illustrate the features of the ureteral modulation wire 206. A variable buoyancy tip may be used on both the irrigation catheter 202 and the ureteral modulation wire 206 to enable movement and flotation within the corresponding kidneys and bladder of the patient. FIG. 11 further describes the features of a variable buoyancy tip.

As shown in FIGS. 2 and 3, the triple lumen nephrostomy tube 204 provides external access to the internal portions of the kidney 102. The fully deployed triple lumen nephrostomy tube 204 enables drainage of the renal pelvis. The irrigation catheter 202 may provide manual irrigation of the kidney's urine collecting system, which can dilute the urine and assist with the breakdown of kidney stones. The irrigation catheter 202 may also assist with chemical degradation of urolithiasis if the proper solutions are used. The ureteral modulation wire 206 may use electrical stimulation via a low-level current to induce cessation of ureteral peristalsis by continuously stimulating the ureter 120. This electrical stimulation may improve pain from arrhythmic contractions of the ureter 120 during an acute episode of renal colic. The stimulation of the ureter 120 over a long period of time may accelerate the accommodation of the device and condition the lumen of the ureter 120 to facilitate passage of the kidney stone.

FIG. 4 shows a cross-section view of a triple lumen nephrostomy tube or catheter 400 according to certain embodiments of the present invention. In some embodiments, the outer surface 402 of the triple lumen nephrostomy tube 400 houses an irrigation catheter 410 and a ureteral modulation wire channel 420. An interior 404 of the triple lumen nephrostomy tube 400 provides an area for drainage of the kidney (not shown). The triple lumen nephrostomy tube 400 may be inserted in the kidney of the patient through a standard of care procedure. Local anesthesia may be applied to the back or posterior of the patient. Then ultrasound imaging or another means may be used to insert a needle that is then replaced by an outer sheath into the proper area of the kidney, wherein the outer sheath will enable insertion of the triple lumen nephrostomy tube 400. Once properly placed in the patient, one end of the nephrostomy tube is located in the kidney of the patient, while the other end remains outside of the body and is connected to a bag to collect urine.

The triple lumen nephrostomy tube 400 not only enables the drainage of urine through the interior 404, but also enables irrigation through the irrigation catheter 410. The irrigation catheter 410 includes an interior 414 portion that allow liquid to flow and an irrigation catheter tip 412 for opening and closing the irrigation catheter 410. When the irrigation tip 412 is open, liquid or water can flow or irrigate the kidney of the patient or other areas, and when the irrigation tip 412 is closed liquid or water cannot flow. The irrigation catheter 410 will be further described herein.

The triple lumen nephrostomy tube 400 further comprises the ureteral modulation wire channel 420 for traveling down the kidney and into the ureter by the ureteral modulation wire (not shown). The ureteral modulation wire channel 420 includes an interior 424 for the insertion, movement, and extraction of the ureteral modulation wire. The ureteral modulation wire channel 420 may also include a ureteral modulation wire tip 422 that signifies the end of the ureteral modulation wire to ensure proper placement. The ureteral modulation wire will be further described herein.

By including an irrigation catheter 410 and a ureteral modulation wire within the triple lumen nephrostomy tube, drainage of the kidney, irrigation of the kidney, and stimulation of the ureter can be accomplished through a single medical procedure. In some embodiments, the outer lumen size 402 may be 8 Fr, while the irrigation catheter 410 is 5 Fr and the ureteral modulation wire channel is 4 Fr. Other sizes and locations of the irrigation catheter 410 and ureteral modulation wire channel 420 are within the scope of the present invention. The outer lumen size of 8 Fr may be chosen because the inner drainage channel should not need to accommodate more than 1.5 L of urine output in a 24-hour period and should have low probability of occlusion secondary to debris or blood products because of the manual irrigation of the collecting system. As will be discussed below, irrigation of the kidneys with a substance that aids in the passage and chemical degradation of urolithiasis may be desired.

The outer sheath (not shown) that is used for placement of the triple lumen nephrostomy tube 400 may include a temperature probe below the surface of the skin to detect and monitor a febrile episode. During a febrile episode, irrigation will be locked out for 48 hours or when cleared by the third-party monitoring system acting as a safety feature to prevent harm to the patient. The temperature probe will be located on the side opposite of the irrigation catheter 410 to prevent a heat sink phenomenon associated with the irrigation of fluids at temperatures dissimilar to endogenous temperatures, but may also be contained external to the body, on the surfaces of the body by any of the other components listed here (i.e., on the catheter). The material used for the outer surface 402 of the triple lumen nephrostomy tube 400 may include a coating both on the inner and outer surface that resists the formation of stone products as this will be placed within the urinary system. Reinforcement of the triple lumen nephrostomy tube 400 to resist extrinsic compression may also be desired to afford consistent drainage, but such reinforcement may compromise patient comfort. In some embodiments, the irrigation catheter 410 may be removed and the modulation wire channel 420 may collapse to enable passage of the larger kidney stones.

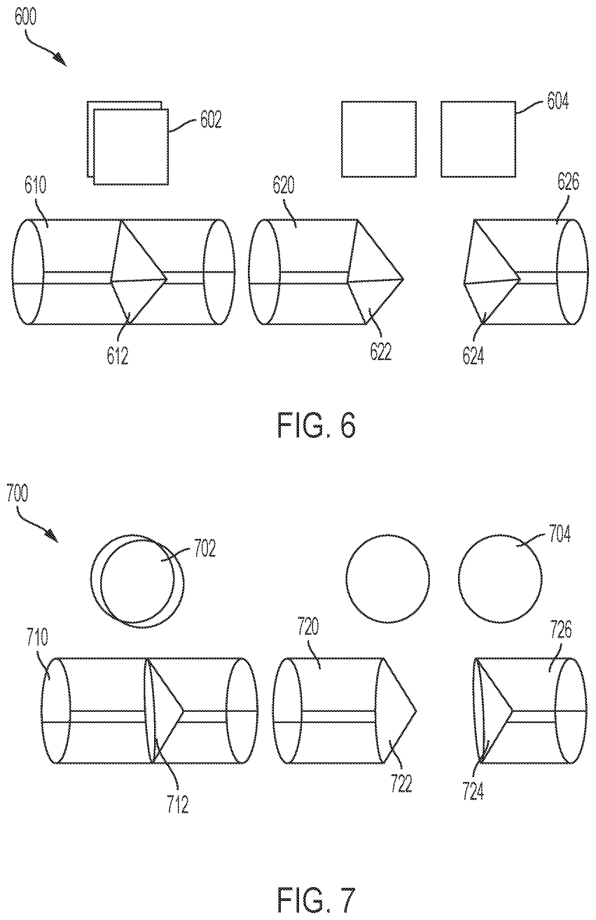

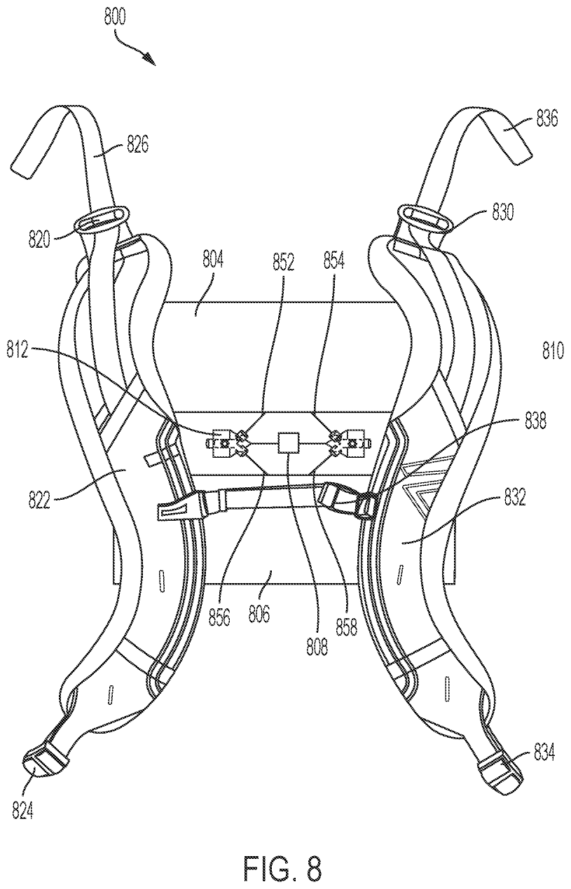

FIG. 5 shows a cross-section view of a left-sided coupling device 500 between a triple lumen nephrostomy tube 400 and external fluid and control connections according to certain embodiments of the present invention. An outflow pressure regulator 502 may control the drainage pressure from a nephrostomy tube 510. The nephrostomy tube 510 may connect to the coupling device 500 through a quick connect interface that will be described with reference to FIGS. 6-7. An irrigation valve 504 may control the irrigation pressure for irrigating a patient's kidney by connecting to the irrigation catheter 410. A tube (now shown) may be used to connect the irrigation valve 504 to an irrigation unit. The procedures for drainage and irrigation will be further described with reference to FIG. 8. A drainage valve 508 may connect to a container for collecting the urine from the patient's kidney. A tube (now shown) may be used to connect the drainage valve 508 to a collection container. Urine will drain from the kidney when the pressure is sufficiently elevated. The irrigation valve 504 and the drainage valve 508 may also include a quick connect interface that will be further described with reference to FIGS. 6-7. In some embodiments, this quick connect interface may use magnetic interfaces. An input connection or interface 506 may provide a connection to an input for ureteral modulation through a ureteral modulation wire in some embodiments. The coupling device 500 may include a battery or other power supply for providing power to the ureteral modulation wire and potentially the outflow pressure regulator 502 and the irrigation valve 504. A caudal surface 520 of the coupling device 500 may adhere to the skin of the patient for easy access. The adhesive surface may be hypoallergenic and suitable for prolonged use. In some embodiments, the coupling device 500 will be attached to the patient's skin at a designated height above or below the target organ to facilitate both drainage and irrigation of the kidneys.

In operation, the nephrostomy tube 510 that has been inserted in the patient's kidney may be connected to the coupling device 500 by the patient. Urine draining from the kidney may then flow through the outflow pressure regulator 502 and out the drainage valve 508 into a collection container or bag. In some embodiments, the outflow pressure may be controlled by the outflow pressure regulator 502 to prevent pyelovenous backflow, which can be unsafe for the patient. For example, a limit of 500 Pa may be set to prevent pyelovenous backflow. Other pressure limits may be selected by the doctor or the patient. For irrigation, the irrigation valve 504 may be connected to a water or solution supply through a tube (not shown). Accordingly, the coupling device 500 will include a pressure sensor for detecting pressure at or near the outflow pressure regulator 502. The irrigation valve 504 can then control the irrigation volume and timing. For example, the irrigation valve 504 may be open for 12 seconds and then closed for 2 minutes to enable proper irrigation to the patient's kidney and proper drainage after irrigation. Other irrigation/drainage time periods may be selected by the doctor or the patient. The outflow pressure regulator 502 may be used to ensure that water flows into the kidney during the 12 seconds of irrigation and drains out of the kidney during the drainage period. Additional valves may also be used to ensure that the irrigation fluid does not flow through the drainage valve 508 and into the collection container during the irrigation period. No irrigation should take place until the pressure has decreased to an unobstructed state, so the coupling device 500 may also stop irrigation when the pressure readings of the outflow pressure regulator 502 are high. And prior to allowing irrigation, there should be no outflow for a predetermined period of time, which can be controlled by a lockout feature within the coupling device 500.

The input connection 506 may incorporate the wiring for the ureteral modulation wire, which will be further described with reference to FIGS. 9 and 10. A corresponding power supply within the coupling device 500 (e.g., battery) or external power supply may power the ureteral modulation wire and control the coupling device 500. A urometer (and corresponding battery or power supply) within the coupling device and a central processing unit or processor may be used to assess the rate of drainage and can be used to communicate with a computer program or application to evaluate in real time when the patient has completed the conservative management of obstructive urolithiasis. This system may need to be calibrated to account for changes in barometric pressure at variable elevations and patients may need to be advised to remain at the same altitude at the time of calibration if a gravity fed system is used for irrigation.

This coupling device 500 was designed to resolve a few drawbacks of prior methods and systems. First, this coupling device 500 provides a first line of defense for inadvertent traction injury to the nephrostomy tube from entanglement of drainage tubing. If entangled, the quick connect will simply disengage and the system will be in a locked/undrained state. Second, this coupling device 500 serves as a continuous evaluation of ureteral obstruction because of the conditional drainage of the collecting system once intrarenal pressures exceed the limit. This facilitates the antegrade passage of urine and stone material through the ureter, thereby reducing the amount of urine that needs to be drained. Third, most nephrostomy tubes cannot be reached by patients. Using this quick connect drainage and irrigation docking stations (FIGS. 6-7), this device can be easily accessed and used by a person living independently.

FIG. 6 shows multiple views of a connection interface 600 for a coupling device between a triple lumen nephrostomy tube and external fluid and control connections according to certain embodiments of the present invention. FIG. 7 shows multiple views of an alternative connection interface 700 for a coupling device between a triple lumen nephrostomy tube and external fluid and control connections according to certain embodiments of the present invention. FIGS. 6 and 7 show the coupled and uncoupled versions of these quick connection interfaces 600, 700 that are designed to be easily accessed, handled, engaged, and disengaged by a patient. Magnetic strips 602 and 604 may be square shaped and designed to easily attach and seal when properly aligned, and easily detach when the magnetic strips 604 are separated. For example, the nephrostomy tube 610 may contain magnetic strips 602 that seal and lock in place when properly aligned with the magnetic strips 602 of the connection interface 612 associated with the coupling device. When the patient decides to detach the nephrostomy tube 620, the corresponding magnetic strips are detached 622 from the magnetic strips 624 of the connection interface 626 associated with the coupling device.

Magnetic strips or rings 702 and 704 may be circle or ring shaped and designed to easily attach and seal when properly aligned, and easily detach when the magnetic strips 704 are separated. For example, the nephrostomy tube 710 may contain magnetic strips 702 that seal and lock in place when properly aligned with the magnetic strips 702 of the connection interface 712 associated with the coupling device. When the patient decides to detach the nephrostomy tube 720, the corresponding magnetic strips or rings are detached 722 from the magnetic strips or rings 724 of the connection interface 726 associated with the coupling device. In some embodiments, the size, shape, and/or orientation of the magnetic strips 602, 604, 702, 704 may vary so that the proper connections are made (i.e., nephrostomy tube to outflow pressure regulator, irrigation valve to water supply, drainage valve to tube or collection container). For example, connection interface 600 may be used for the irrigation valve connection and connection interface 700 may be used for the drainage valve connection to ensure that the proper connections are made by the patient. Different size connection interfaces may also be used to ensure proper connections. Additionally, the power supply from the ureteral modulation unit (not shown) can be used to provide an electromagnetic charge, and disengage by altering the electromagnetic charge to reduce excessive force placed on the coupling device.

FIG. 8 shows a harness system 800 for enabling a patient to efficiently transport and access external fluid and control connections to a triple lumen nephrostomy tube according to certain embodiments of the present invention. Shoulder straps 822, 832 make up the harness that is designed to worn around the shoulders of the patient in order to hold the necessary components of the harness system 800. Lower adjustment buckles 824, 834, upper adjustment buckles 820, 830, and upper adjustment straps 826, 836 are designed to hold the harness system 800 in place and provide adjustment flexibility for the patient. More specifically, the patient can adjust the harness system 800 for the desired fit through use of the adjustment straps 826, 836 and corresponding adjustment buckles 824, 834, 820, 830. A chest strap 838 may be used to keep the shoulder straps 822, 832 in place. An irrigation unit 804 provides the water or solution supply for the irrigation features mentioned above, while a drainage unit 806 provides a collection container for the urine drainage features mentioned above. The irrigation unit 804 is higher on the patient to allow gravity to assist with irrigation into the kidneys, and the drainage unit 806 is lower on the patient to allow gravity to assist with urine drainage from the kidneys. In addition to using gravity, the irrigation unit 804 may be pressurized to assist with irrigation. Two coupling devices 810, 812 are shown for both kidneys. If the harness system 800 is only being used on one kidney, then only one coupling device 810, 812 may be required. As mentioned above, the coupling devices may be firmly adhered to the skin of the patient for easier control of the tubes and units involved. In some embodiments, the shoulder straps 822, 832 are worn on the front of the patient with irrigation unit 804 and drainage unit 806 on the back of the patient, similar to a backpack.

A first irrigation tube connection 852 connects the irrigation valve of the coupling device 812 to the irrigation unit 804, and a second irrigation tube connection 854 connects the irrigation valve of the coupling device 810 to the irrigation unit 804. A first drainage tube connection 856 connects the drainage valve of the coupling device 812 to the drainage unit 806, and a second irrigation tube connection 858 connects the drainage valve of the coupling device 810 to the drainage unit 806. These interfaces and connections may be similar to those described with reference to FIGS. 6-7 to enable quick connection and detachment by the patient. Once the harness system 800 and the coupling devices 810, 812 have engaged, there will be a pressure gradient system that takes into account the internal hydrostatic pressure of the hollow visceral structure of the kidneys as well as the gravitational direction of both the irrigation unit 804 and the drainage unit 806. The drainage unit 806 may be positioned close to the surface of the patient's skin to reduce the occurrence of unintentional coupling, unintentional removal of the nephrostomy tube, and reduce the centrifugal force of fluid within the drainage unit 806. The irrigation unit 804 may be positioned close to the skin for similar reasons. The harness system 800 also includes a ureteral modulation unit 808 that can control the operation of the ureteral modulation wire, provide power to the system, and control other components of the present invention (i.e., coupling devices). The ureteral modulation unit 808 may be connected to the coupling devices 810, 812 through an input connection 506 (FIG. 5). The ureteral modulation unit 808 may also be firmly adhered to the skin of the patient at a central area of the patient, possibly over the spine.

The harness system 800 is designed to be removable and adjusted by the patient. Docking the harness system 800 and coupling devices 810, 812 can be illustrated to the patient with a signal showing “docked” and “undocked” state using a green and red light illuminated at the level of the chest strap 838, so that it is visible to the patient. Input controls at the chest strap 838 level of the patient may also be used to disengage the coupling devices 810, 812 from the harness unit 800. This harness system 800 can be used during both nocturnal and diurnal activities. Different size irrigation units 804 and drainage units 806 may be used based upon duration and volume of urine output, and larger size drainage units 806 may be required for nocturnal use. The irrigation unit 804 and drainage unit 806 may be disposable, while the remaining portions of the harness system 800 may be reusable.

For night drainage, the drainage unit 806 may be a bag with a larger surface area to prevent a significant increase in the depth of the drainage bag as it fills. There may be an internal valve system with a preferential gravity feed depending upon the expected sleeping position of the patient to allow for multiple sleeping positions without the retrograde flow of urine. The bag may be reinforced to prevent rupture in the event that undue pressure is placed on the drainage bag, with some degree of elasticity built into the bag to reduce the traumatic force applied to the body in the event of an abrupt contact with a fixed object, or rapid deceleration or acceleration injury. There should also be a layer between the surface of the skin and the drainage unit 806 to prevent any skin breakdown created by an anerobic environment or caustic agent.

FIG. 9 shows a ureteral modulation unit 900 according to certain embodiments of the present invention. A housing 902 for the ureteral modulation unit 900 may house at least a central processing unit or processor 904, a battery or power supply 906, and a wireless transceiver 908 for potential wireless communications with other components. The ureteral modulation unit 900 may have a grounding unit built in, which will connect to the ureteral modulation wire (FIG. 10). The battery or power supply 906 may provide power to the ureteral modulation unit and the coupling device. As shown in FIG. 8, the ureteral modulation unit 900 may be connected to a first coupling device through connection line 910 and a second coupling device through connection line 920. Ureteral modulation unit 900 may also support connection to an external power supply (not shown) or connection to a mobile device or computer to upload or download data. Through the wireless transceiver 908, the ureteral modulation unit 900 may transmit and receive data from a peripheral mobile device or computer within close proximity. The peripheral device (not shown) may contain a display unit that can communicate with the patient. Through this connection, the present invention can be monitored and controlled by the patient or even by a third party via the internet. Thus, symptoms and observations may be appreciated and applied to care for the patient. Modulation unit 900 may also be located within the coupling device to provide processing and battery power to the valves and components of the coupling device.

FIG. 10 shows a ureteral modulation wire 1000 according to certain embodiments of the claimed invention. The body of the ureteral modulation wire 1002 terminates at a first end of the ureteral modulation wire 1030. Thus, the doctor can insert the wire into the patient, through the kidney, and into the desired position in the ureter, at the ureterovesicular junction, or within the bladder. The first end 1030 may include a sensor, RFID tag, or other type of location component to assist the doctor with placing it in the right position of the ureter of the patient. The first end may 1030 also be buoyant so that floats in the bladder of the patient and does not contact the trigone. A first set of electrodes 1010, 1014 are placed at intervals along the ureteral modulation wire 1000 for the detection of muscular contraction during peristalsis. These first electrodes 1010, 1014 are responsible for detecting ureteral peristalsis through an electromyogram. A second set of electrodes 1012, 1016 within the ureteral modulation wire 1000 provides for the transmission of electrical current. These second electrodes 1012, 1016 are capable of monopolar, bipolar, or other current to either induce ureteral peristalsis or paralyze the ureter preventing ureteral peristalsis. The second set of electrodes 1012, 1016 may be fired in sequential fashion to propagate ureteral peristalsis. The number of leads and electrodes within the ureteral modulation wire 1000 may be from 1-100.

Between the electrodes 1010, 1012, 1014, 1016 are a set of reinforced tubing 1020, 1022, 1024 that are capable of radial expansion. Radial expansion of these tubing sections 1020, 1022, 1024 may be achieved by expanding the material making up the tubing (i.e., wire mesh that can expand) or simply applying pressure to both sides of the tubing sections to radially expand the tubing through compression. Pneumatic (applying air), hydrostatic, mechanical, or other means are within the scope of the present invention for radially expanding the tubing. Prolonged exposure of the ureter to this ureteral modulation wire 1000 and corresponding expansion of the wire 1000, induces an accommodation effect, thereby increasing the diameter and pliability of the ureter, thus facilitating the passage of solid objects. Pharmacological compounds or coatings may be applied to the ureteral modulation wire 1000 to improve pain, modulate the ureter, or prevent infection or further complications.

FIG. 11 shows an irrigation catheter 1100 according to certain embodiments of the claimed invention. As previously discussed, an irrigation catheter 1100 is designed to irrigate the kidney with saline, liquid, or a desired solution. After insertion into the kidney, the irrigation catheter 1100 may need to travel to various portions of the kidney for proper irrigation. Thus, the length of the irrigation catheter 1100 should be chosen to facilitate movement within the kidney. A variable buoyancy tip 1104 is connected to the body of the irrigation catheter 1106. A channel 1108 runs through both the body of the irrigation catheter 1106 and the variable buoyancy tip 1104 for the liquid or water to flow into the kidneys for irrigation. A surface 1110 of the buoyancy tip 1104 may be used to assist with movement and location of the irrigation catheter 1100. For example, sensors or RFID tags on the surface 1110 may be used to identify the movement and location of the irrigation catheter 1106.

The variable buoyancy tip 1104 allows migration of the irrigation catheter 1100 through each calyx of the kidney. This buoyancy may be controlled or managed through connection to the coupling device or the ureteral modulation unit. Pneumatic, hydraulic, and/or electrical means may provide the necessary variation for the buoyancy tip 1104. For example, air could be used to fill up the buoyancy tip 1104 such that the additional water provided by the irrigation catheter 1100 would cause flotation to the top of the kidney. Electrical current applied to a motor-type component may also combine with the flotation feature to enable travel to any portion of the kidneys. The irrigation catheter 1100 would be used to carry substances, air, or electrical current to the variable buoyancy top 1104. The use of electrical current would likely provide the smallest change in structure of the irrigation catheter 1100, whereas hydraulic or pneumatic means would require dedication of a portion of the triple lumen nephrostomy tube to the irrigation catheter 1100 to achieve a change in the buoyance of the tip as well as protection from impingement of this tubing. The goal is to allow the irrigation catheter 1100 to provide irrigation to all portions of the kidneys.

The diameter of the combined triple lumen nephrostomy tube would have to be larger than the maximum diameter of the variable buoyancy tip 1104 such that an accidental removal of the triple lumen nephrostomy tube would not apply pressure to the patient's kidney. An ideal solution would be a chamber within the variable buoyance tip 1104 that expands or contracts in length rather than girth. Another safety feature of the present invention may be preventing or locking out irrigation if the drainage portion of the triple lumen nephrostomy tube is removed or is not functioning. Accordingly, the coupling device may lock out irrigation if urine drainage is unavailable. Irrigation may only occur during waking hours and may stop two hours before planned or scheduled sleep times.

Irrigation of all areas of the kidney will help improve stone clearance rates after extracorporeal shock wave lithotripsy and may be used as an adjunct to this form of surgical management of urolithiasis. Irrigation alone provides mechanical propulsion of solid objects within the collecting system and increases the natural rate of peristalsis. An alternative option to variable buoyance would be a nozzle that directs the flow of the irrigation fluid that can be used to propel the distal aspect of the irrigation tubing as a form of navigation such that each calyx within the entire collecting system will be entered and irrigated by the irrigation catheter.

The ideal solution to be used as the irrigation solution or liquid in the present invention will depend upon the patient and the type of stone material. The variables to be used in the irrigation fluid may include altering the pH, mineral composition (sodium, chloride, potassium, citrate, etc.), pharmacological agents, and temperature as these are all possible variations that the urinary system has evolved to accommodate under normal physiological conditions. Other potential substances to be included in the irrigation fluid may include inhibitors of stone formation. Directly inserting these inhibitors into the collecting system in a safe and effective manner may possibly achieve supraphysiological parameters that could never be achieved through enteric or parenteral administration of pharmacological agents. Markers may also be used in the irrigation solution to determine how urine is passing through the kidneys, ureters, and bladder. For example, a doctor may be able to determine if a ureter or portions of the kidney are obstructed by irrigating with a marker and then observing the urine that passes through the bladder.

The present invention may include synchronized data transmission of all data points (i.e., volume of urine, volume of irrigation fluid, temperature, ureteral peristalsis, radial expansion of ureteral modulation wire, etc.) to a centralized processing unit, mobile device, or software. This can be done through aggregation by a central processing unit of the ureteral modulation unit and wireless transmission to this central processing unit or processor, mobile device, or software. A peripheral device or mobile device may provide intermediate aggregation or analysis before transmission to the centralized processing unit or through the internet. In this fashion, the various components of the present invention may be synchronized, sequenced, monitored, and controlled by a third party. In the event that a safety event triggers irrigation to be halted, the central processing unit may need to be in communication with the peripheral device or mobile device in order to resume irrigation. Drainage may not need to be controlled by the central processing unit or the peripheral device, although these devices may track corresponding volume. Data from the various components of the present invention may be analyzed by software and/or artificial intelligence to drive treatment algorithms that will be coupled with patient driven outcomes.

As discussed above, numerous safety features will be in place to prevent harm to patients. Protocols should be in place to ensure that the kidney has been decompressed for a defined period of time to allow for clearance of underlying infections and any necessary cultures have returned. After an assessment of infection is declared negative, irrigation may commence. As mentioned above, a temperature probe may be built into the nephrostomy tube that will transmit the core temperature of the patient. The patient should be afebrile for greater than 48 hours and will need to be in communication with the computer program, software application, or other third party to ensure that no symptoms exist that that irrigation may resume. Once cleared to resume, a signal may be received from the computer programs, software application, or third party to allow irrigation to commence. Another safety feature will be the assured low intrarenal pressure state. Because the coupling device allows for selective outflow based on elevated intrarenal pressures, the irrigation of the urinary system will be conditional on a low-pressure state. This can also be confirmed with a manometer included in either the distal aspect of the drainage catheter, intrarenal portion of the ureteral modulation wire, or the distal aspect of the irrigation catheter. In order to reduce or prevent trauma from the inadvertent removal of the entire system, it is mandatory that no portion of these devices are fixed within the body to allow for atraumatic rapid removal with traction applied to the nephrostomy tube and the internal components within. The diameter of all internal components must be of uniform length to prevent injury to the patient. All components may be manufactured to ensure that fracturing of the device with subsequent deposit of foreign substances in the body will not occur.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A medical device for use with a patient's urinary tract, comprising:
a coupling device that comprises a first connection interface that is configured to be connected to a nephrostomy tube that is configured to be medically inserted into a kidney of said patient, and a second connection interface that is configured to be connected to an irrigation unit that is configured to hold a liquid;
an irrigation catheter that is configured to be controlled by said coupling device to adjust a flow of said liquid to said kidney of said patient, wherein at least a portion of said irrigation catheter is further configured to be housed in said nephrostomy tube; and
an irrigation valve that is configured to control said flow of said liquid through said irrigation catheter.

2. The medical device of claim 1 wherein said coupling device further comprises a third connection interface that is configured to be connected to a drainage unit that is configured to enable urine from said kidney of said patient to drain into said drainage unit.

3. The medical device of claim 2 wherein said coupling device further comprises a drainage valve that is configured to control said flow of said urine to said drainage unit.

4. The medical device of claim 3 wherein said coupling device further comprises an outflow pressure regulator that is configured to monitor a drainage pressure of said patient's kidney.

5. The medical device of claim 4 further comprising a processor that is configured to control said irrigation valve and said drainage valve in response to said drainage pressure monitored by said outflow pressure regulator.

6. The medical device of claim 3 wherein said coupling device further comprises a processor that is configured to control said irrigation valve and said drainage valve.

7. The medical device of claim 1 wherein said coupling device further comprises a ureteral modulation unit that is configured to control a ureteral modulation wire.

8. The medical device of claim 1 wherein said first connection interface and said second connection interface are configured to be manually attached and detached by said patient.

9. The medical device of claim 1 wherein said first connection interface is further configured to be connected to a triple lumen nephrostomy tube that is configured to house at least a portion of said irrigation catheter and at least a portion of a ureteral modulation wire.

10. The medical device of claim 1 wherein said irrigation catheter has a variable buoyancy tip.

11. A system for the medical treatment of a patient's urinary tract, comprising:
a coupling device that comprises a first connection interface that is configured to be connected to a nephrostomy tube and a second connection interface that is configured to be connected to an irrigation unit that is configured to hold a liquid;
an irrigation catheter that is configured to be controlled through said coupling device, wherein at least a portion of said irrigation catheter is further configured to be housed in said nephrostomy tube;
a ureteral modulation wire that is configured to be inserted into said patient through said nephrostomy tube; and
an irrigation valve that is configured to control said flow of said liquid through said irrigation catheter.

12. The medical device of claim 11 wherein said coupling device further comprises a third connection interface that is configured to be connected to a drainage unit.

13. The medical device of claim 12 wherein said coupling device further comprises a drainage valve that is configured to control a flow of urine of said patient to said drainage unit.

14. The medical device of claim 13 further comprising a central processing unit that is configured to control said irrigation valve and said drainage valve.

15. The medical device of claim 11 wherein said ureteral modulation wire is further configured to monitor a muscle contraction of a ureter of said patient.

16. The medical device of claim 11 wherein said ureteral modulation wire is further configured to radially expand in a ureter of said patient.

17. A medical device for the treatment of a patient's urinary tract, comprising:
a coupling device that comprises a first connection interface that is configured to be connected to a nephrostomy tube that is configured to be medically inserted into a kidney of said patient, a second connection interface to an irrigation unit that is configured to hold a liquid, and a third connection interface to a drainage unit that is configured to collect urine from said patient's kidney;
wherein said first connection interface is further configured to be connected to an irrigation catheter that is configured to irrigate said kidney of a patient, wherein at least a portion of said irrigation catheter is further configured to be housed in said nephrostomy tube;
wherein said coupling device further comprises an irrigation valve that is configured to control a flow of said liquid through said irrigation catheter and a drainage valve that is configured to control said collection of said urine to said drainage unit.

18. The medical device of claim 17 wherein said coupling device further comprises an outflow pressure regulator that is configured to monitor a drainage pressure of said patient's kidney.

19. The medical device of claim 17 further comprising a processor that is configured to control said irrigation valve and said drainage valve.

20. The medical device of claim 17 wherein said first connection interface is configured to be manually attached and detached by the patient.
